(19) United States
(12) Patent Application Publication
(10) Pub. No.: US 2008/0300172 A1
(43) Pub. Date: Dec. 4, 2008

(54) Method and Compositions for the Treatment of Diabetes and Related Complications

(75) Inventor: Samuel K. Yue, Edina, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Sky BioHealth Solutions, Inc.

(21) Appl. No.: 12/116,342

(22) Filed: May 7, 2008

Related U.S. Application Data
(63) Continuation of application No. 11/438,466, filed on May 22, 2006, now abandoned.
(60) Provisional application No. 60/689,954, filed on Jun. 13, 2005.

(51) Int. Cl. A61K 38/28; A61K 38/22; A61P 3/10

(52) U.S. Cl. 514/4; 514/3; 514/12

(57) ABSTRACT

The present invention relates to administration of relaxin and related polypeptides to treat a variety of conditions, including diabetes, diabetes-related conditions, Alzheimer's disease, and menopause and related conditions.

### CROSS-REFERENCE TO RELATED APPLICATION(S)

This application is a continuation of U.S. patent application Ser. No. 11/438,466, filed May 22, 2006, entitled “Method and Compositions for the Treatment of Diabetes and Related Complications”, which claims the benefit of U.S. Provisional Application No. 60/689,954, entitled “Method and Compositions for the Treatment of Type I and Type II Diabetes and Related Complications,” filed on Jun. 13, 2005, which are both hereby incorporated herein by reference in their entirety.

### FIELD OF THE INVENTION

The present invention relates to administration of relaxin and related polypeptides to treat a variety of conditions, including diabetes, diabetes-related conditions, Alzheimer's disease, and menopause and related conditions.

### BACKGROUND OF THE INVENTION

Dr. Hisaw, the founder and forerunner on the field of endocrinology, first discovered relaxin, an insulin-like polypeptide, in 1927. Known as the third major pregnancy hormone, relaxin has been studied extensively from the 1930s to the present and has been determined to have multiple physiological functions besides those related to pregnancy. Study of humans indicate that relaxin is secreted in a pulsatile nature in females. This usually is measurable in the blood stream approximately during the menstrual mid-cycle surge of the luteinizing hormone or approximately seven to ten days after ovulation and if conception occurs, relaxin continues to rise to over 800 picograms per ml by the third week. In a normal menstrual cycle, secretion of relaxin peaks at about 80-100 picograms and lasts approximately 3-5 days.

Relaxin hormone has been prepared from animals, particularly from the ovaries of pregnant sows, and was used quite extensively in the 1950s and 1960s as an agent for shortening labor, ripening the cervix, and treating scleroderma and peripheral vascular disease. It was an FDA-approved medication until 1972 when it and a whole host of other drugs were taken off the market because of the Kefauven-Harris Drug Amendment Act passed by the US Congress in 1962.

Diabetes is a disease in which the body does not produce or properly use insulin. An estimated 20.8 million children and adults in the United States have diabetes. Another 41 million people are estimated to have “pre-diabetes,” a condition which is typically a precursor to type 2 diabetes and is exhibited by blood glucose levels that are higher than normal but not yet high enough to be diagnosed as diabetes. About 1.5 million new cases of diabetes were diagnosed in people aged 20 years or older in 2005.

There is a need in the art for a new treatment for diabetes and diabetes-related conditions.

### BRIEF SUMMARY OF THE INVENTION

The present invention, in one embodiment, is a method for treating diabetes-related conditions in a subject. The method includes administering relaxin to the subject, whereby the subject's supplemental insulin requirements are reduced. According to one embodiment, the diabetes-related condition is chosen from the group consisting of pre-diabetic syndrome, Type II diabetes, Type I diabetes, and Alzheimer's disease.

In another embodiment, the present invention is a method for treating menopause-related conditions in a subject. The method includes administering relaxin to the subject, whereby the subject's menopause-related conditions is improved.

The present invention according to another embodiment is a method of anti-aging treatment. The method includes administering relaxin to a subject, whereby the subject's insulin resistance is decreased.

In accordance with a further embodiment, the present invention is a method for treating diabetes-related conditions in a subject, comprising suggesting administration of relaxin to the subject. The method can also include administering relaxin to the subject, whereby the subject's supplemental insulin requirements are reduced. In an additional embodiment, the method includes suggesting administration of insulin, and further can include administering insulin.

The present invention, according to one aspect of the invention, is a kit for treating a condition in a subject. The kit includes a dosage of relaxin and instructions for administration of the relaxin.

### DETAILED DESCRIPTION OF THE INVENTION

The present invention relates to a method and composition for treatment of Type I and Type II Diabetes and Alzheimer's disease and complications related to these diseases. More specifically, the present invention relates in one aspect to administering relaxin hormone or a combination relaxin-insulin hormone or related polypeptides or analogs to a patient for treatment of various conditions and diseases, including Type I and Type II diabetes and Alzheimer's disease and related complications. The present invention further relates to methods and compositions for use in hormone replacement therapy and treatment of other conditions as specified herein.

### Treatment of Type I and Type II Diabetes and Alzheimer's Disease and Related Complications

According to one embodiment, the hormone relaxin and related polypeptides and analogs may be administered to a patient to treat Type I diabetes, Type II diabetes, diabetes-related complications, or Alzheimer's disease. Alternatively, both relaxin and insulin may be administered simultaneously to treat any of the same conditions. In a further alternative of the present invention, a hybrid polypeptide comprising both relaxin and insulin functions may be administered to treat any of the same conditions.

### Treatment of Type I or Type II Diabetes with Relaxin

Administration of relaxin, in one aspect of the present invention, may delay the onset of insulin resistance in patients with Type II diabetes, which can also be referred to as adult onset diabetes (“AODM”). Further, a patient with pre-diabetic syndrome may be administered relaxin to delay the onset of clinical diabetes. In another embodiment, in patients with Type II diabetes in a relatively advanced state of the disease in which insulin supplementation is required, relaxin may be administered to reduce the patient's resistance to insulin by reducing the cellular resistance to insulin and thereby to reduce the patient's supplemental insulin requirement and to delay the onset of a more advanced state of the disease. Relaxin may also be administered to extremely advanced Type II diabetes patients with high insulin resistance (also referred to as “brittle” diabetic patients) to reduce insulin resistance and normalize glucose levels in the blood. Alternatively, the administration of relaxin may reverse the symptoms of Type II diabetes in a patient.

Administration of relaxin, according to another embodiment of the invention, may delay the need for progressively greater amounts of supplemental insulin in patients with Type I diabetes. Further, a patient with Type I diabetes may be administered relaxin to delay the onset of a more advanced state of the disease. Alternatively, the administration of relaxin may reverse the symptoms of Type I diabetes in a patient.

According to one embodiment, administration of relaxin to a patient can result in the patient's supplemental insulin requirement drop by 40-50%, stabilize the patient's daily glucose level, reduce the patient's HbA1c level to within normal levels, reduce the creatine level of any patient with a renal insufficiency, and produce subtle improvements in other of the patient's conditions relating to Type I or Type II diabetes.

Relaxin, according to one embodiment of the present invention, decreases cell resistance to insulin. Studies have shown that relaxin decreases the insulin resistance of female mice fatty cells. See, for example, “Potentiation of insulin binding and insulin action by purified porcine relaxin,” by Olefsky J M; Ann N Y Acad Sci., 1982, which is incorporated herein by reference in its entirety. Without being limited by theory, it is theorized that relaxin can decrease cell resistance to insulin because both relaxin and insulin follow a common pathway through the cell wall into the cell body. As a result, activating the receptor site with one hormone will increase the affinity or decrease the resistance of the other receptor site to the other hormone. Thus, relaxin can activate its own receptor site in the cell wall and thereby increase the affinity or decrease the resistance of insulin receptor sites to the insulin molecule, thereby reducing the amount of insulin needed to bind with the receptor site in order for serum glucose to be able to easily penetrate the cell wall into the cell body. The ultimate effect is a decreased demand for insulin placed on the pancreas. Thus, it is theorized herein that administration of relaxin decreases cell resistance to insulin.

Further, administration of relaxin to insulin-dependent diabetic patients in accordance with one aspect of the invention results in decreased supplemental insulin requirements for those patients. More specifically, clinical observations have shown that administration of relaxin to certain insulin-dependent diabetes patients has resulted in those patients' supplemental insulin requirements decreasing by about 40% to about 50%. Without being limited by theory, it is believed that the decrease in the insulin requirements of insulin-dependent patients may be caused by the impact of relaxin on cell resistance to insulin. That is, as the cell resistance to insulin decreases as a result of relaxin, less insulin is required and the insulin-dependent patient requires fewer or smaller insulin injections.

In accordance with another embodiment of the present invention, administration of relaxin to a diabetic patient can result in the decrease in the level of Hemoglobin A1c (“HbA1c”) in the blood. Without being limited by theory, this decrease may be caused by relaxin causing a decrease in cell wall insulin resistance, which in turn results in a normalization of the glucose level and thereby results in a decrease in the A1C level. HbA1c is a component of hemoglobin to which glucose is bound. The more glucose present in the blood, the more HbA1c is present as well. Given that HbA1c levels in the blood depend on the concentration of glucose in the blood, HbA1c levels can be used as an indicator of blood glucose levels. Thus, it is believed that the administration of relaxin can ultimately result in a decrease in glucose levels in the blood of the patient and the decrease in HbA1c levels are an indication of the decrease in glucose levels.

### Treatment of Diabetes-Related Complications with Relaxin

In addition, according to one embodiment, relaxin may be administered to improve various complications resulting from Type I and Type II diabetes, such as diabetic-related cardiovascular disease, diabetic-related renal disease, diabetic-related retinopathy, diabetic-related pulmonary hypertension from fibrosis, diabetic-related blood pressure elevation, diabetic-related bowel-related problems such as spastic colon and irritable bowel, Type II-related osteoarthritis, Type II-related bone weakness or osteoporosis, rapid aging caused by diabetes, and any other known condition resulting from Type I or Type II diabetes. See, for example, the articles listed in Appendix A, all of which are incorporated herein by reference in their entirety.

According to one embodiment, relaxin may be administered to treat high blood pressure associated with diabetes. Relaxin activates the nitric oxide pathway, thereby causing the smooth muscles within the body to relax. A relaxin deficit results in reduced activation of the nitric oxide pathway, thereby causing a decrease in smooth muscle relaxation. The decrease in smooth muscle relaxation can, according to one aspect of the invention, cause an increase in the blood pressure of many diabetics.

In another embodiment, relaxin may be administered to treat elevated cholesterol associated with diabetes. Relaxin plays a role in the mechanism related to lipid metabolism. A relaxin deficit can cause abnormal lipid metabolism in the patient's body, thereby causing elevated cholesterol and triglyceride levels.

Administration of relaxin, according to one embodiment, may treat fibrosis related to diabetes. That is, relaxin modulates the production of collagen such that it up-regulates collagen in areas in which collagen has been inadequately produced and down-regulates collagen in areas in which too much collagen has been produced. Thus, an overall relaxin deficit can result in generalized and progressive fibrosis of the organs or tissues. Administration of relaxin can treat fibrosis, including arterial hardening of the blood vessels, pulmonary vasculatures, hardening of the organs, such as the heart or kidney, bowel-related problems, and other known problems caused by fibrosis.

In one aspect of the present invention, administration of relaxin can delay, arrest, or reverse the effects of AODM-related osteoporosis or bone weakness, thereby reducing the incidence of bone fractures. As discussed above, relaxin modulates the production of collagen, which is one of the major components of bone structure in the form of the collagen matrix in bone. The collagen matrix functions to bind calcium in the bone structure. It is theorized that administration of relaxin may strengthen the collagen matrix in bone structure and thereby strengthen bones and delay, arrest, or reverse the effects of AODM-related osteoporosis.

In another aspect of the present invention, administration of relaxin may be used to treat conditions related to AODM or Type II diabetes. The relaxin can delay, arrest, or reverse the effects of AODM-related osteoarthritis, which is also referred to as degenerative joint disease (“DJD”). The collagen matrix discussed above operates to provide resiliency to joint cartilage. Strong and resilient joint cartilage can delay early breakdown of the joint. Thus, it is theorized that relaxin administration will strengthen the collagen matrix in joint cartilage and thereby delay, arrest, or reverse DJD.

Relaxin, in accordance with an alternative aspect of the present invention, may be administered to preserve cardiac vasculature and reduce cardiovascular complications, including shortness of breath. Further, administration of relaxin according to another embodiment, can increase normal kidney function in patients with diabetes-related kidney complications. In addition, according to one aspect, administration of relaxin can decrease the elevated level of creatinine in patients suffering a renal deficiency as a result of diabetes.

In another embodiment, relaxin may be administered to treat rapid “aging” associated with diabetes. It is commonly understood that a diabetes patient appears to age about 30% faster than a healthy individual on average. That is, overall body appearance and organ functions deteriorate faster in a diabetes patient in comparison to a healthy individual. Some anti-aging researchers believe that a reduction in a patients' cell wall insulin resistance can slow the physical aging or deterioration process or improve longevity or overall health. Given that relaxin administration can reduce cell wall insulin resistance, it is theorized herein that relaxin can delay the physical aging or deterioration process or improve longevity or overall health.

According to one embodiment, a relaxin-like factor can be administered to treat any of the conditions described above. One non-limiting example of a relaxin-like factor is RLX7. Alternatively, any known relaxin-like factor can be administered. Relaxin and relaxin-like factor operate at the same receptor sites in the human body. In one aspect of the invention, administration of a relaxin-like factor to a male patient may be more effective in treating the male patient's condition or conditions than relaxin. Without being limited by theory, it is theorized that relaxin-like factor may be more effective than relaxin in male patients because as adult males have higher levels of relaxin-like factor than females. That is, male and female pre-pubescent children have similar levels of relaxin, relaxin-like factor, testosterone, estrogen, and progesterone. However, as children pass through puberty, testosterone and relaxin-like factor increase in males, while estrogen, progesterone, and relaxin increase in females. Thus, it is theorized that relaxin-like factor is effective in males because males have a naturally higher level of relaxin-like factor in comparison to females. Alternatively, a combination of relaxin and relaxin-like factor can be administered to a patient.

### DEFINITIONS

For purposes of this application, the term “relaxin” encompasses the entire family of relaxin and its related polypeptides, including, but not limited to, the relaxin hormone, including human relaxin hormone, porcine relaxin hormone, and any other known animal relaxin polypeptide, relaxin-like factors and relaxin-like polypeptides, any analogs of relaxin and related polypeptides, and any similar polypeptides that express the same activity as relaxin. Relaxin analogs can include, but are not limited to, any relaxin polypeptide that is manipulated or altered in any known fashion to increase or strengthen the activity of the polypeptide.

“Administration” of relaxin relates to providing relaxin to a subject or patient. Administration includes administering whole relaxin polypeptides or some portion thereof. Routes of administration include, but are not limited to, oral and parenteral routes, such as intravenous (IV), subcutaneous, transcutaneous, pulmonary, intraperitoneal (IP), rectal, topical, ophthalmic, nasal, and transdermal, or any other known method of administration to a patient. According to one embodiment, relaxin can be administered orally because the need for relaxin is not immediate and thus can be absorbed slowly through the normal digestive process. If administered orally, the relaxin may be provided or administered in the form of a unit dose in solid, semi-solid, or liquid dosage form such as tablets, pills, powders, liquid solutions, or liquid suspensions. Further, relaxin may be administered intravenously in any conventional medium for intravenous injection, such as an aqueous saline medium, or in a blood plasma medium. The medium also may contain conventional pharmaceutical adjunct materials or carriers, such as pharmaceutically acceptable salts to adjust the osmotic pressure, lipid carriers (e.g., cyclodextrins), proteins (e.g., serum albumin), hydrophilic agents (e.g., methyl cellulose), detergents, buffers, preservatives, and the like to increase, delay, or prolong the absorption or to avoid destruction caused by the digestive process. A more complete explanation of acceptable pharmaceutical carriers can be found in Remington: The Science and Practice of Pharmacy (19^{th} Edition, 1995) in chapter 95.

### Treatment of Diabetes or Related Complications with Relaxin and Insulin

According to another embodiment of the present invention, both relaxin and insulin can be administered simultaneously to treat Type I or Type II diabetes and related complications and conditions as described above. That is, both polypeptides can be administered together in the same dosage. The combination of both polypeptides can have the same effect or, according to one aspect of the invention, a more powerful effect, as to the same diabetes and diabetes-related conditions and diseases as described above. As explained above with relaxin, the term “insulin” encompasses the entire family of insulin and its related polypeptides, including, but not limited to, the insulin hormone, including any known animal insulin polypeptides, insulin-like factors, any analogs of insulin and any similar polypeptides that express the same activity as insulin. Further, insulin can be administered by any known method and in any known form as described above with respect to relaxin.

### Treatment of Diabetes or Related Complications with Insulaxin

In a further embodiment, Type I or Type II diabetes patients can be treated with a hybrid polypeptide molecule comprising a combination of insulin and relaxin. This hybrid hormone was first described and created by Dr. Christian Schwabe and can also be referred to as “insulaxin.” See, for example, the article entitled “Chemical synthesis of a Zwitterhormon, insulaxin, and of a relaxin-like bombyxin derivative,” written by E. E. Bullesbach, B. G. Steinetz, and Christian Schwabe, which is incorporated herein by reference in its entirety. As explained by Dr. Schwabe, this hybrid hormone expresses both relaxin and insulin activity in a single molecule. Treating Type I or Type II diabetes patients with insulaxin can have the same effect or, according to one aspect of the invention, a more powerful effect than relaxin alone, as to the same diabetes and diabetes-related conditions and diseases as described above. As explained above with relaxin, the term “insulaxin” encompasses insulaxin and its related polypeptides, including, but not limited to, insulaxin-like factors, any analogs of insulaxin, and any similar polypeptides that express the same activity as insulaxin.

According to one embodiment, insulaxin can be administered first orally to prevent the onset of metabolic disease or Syndrome X, which is described below. Subsequently, if and when the patient develops insulin-dependent diabetes, insulaxin can be administered either orally or intravenously to eliminate or prevent or delay the onset of diabetes and related complications. Alternatively, insulaxin can be administered by any known method and in any known form as described above with respect to relaxin.

### Theory Relating to Effectiveness of Relaxin

While not limited by theory, it is theorized herein that relaxin has a substantial impact on the development of both Type I and Type II diabetes, and that a relaxin deficit may in fact be the root cause of those diseases. The premise of this theory is that diabetes is a two-hormone disease. That is, both insulin and relaxin have a substantial impact on both types of diabetes.

This two-hormone theory will first be discussed in the context of Type II diabetes. Prior to setting forth the theory, a general description of Type II diabetes, including the onset of Type II, is set forth. The patient generally first enters a pre-diabetic state, which is also referred to as “metabolic syndrome” or “Syndrome X.” Metabolic syndrome (global change) or Syndrome X is a precursor condition to diabetes such that a person with Syndrome X is at risk to develop Type II diabetes. The onset of Syndrome X is insulin resistance. That is, the cell walls of the cells in the patient's body begin to develop resistance to the action of the insulin. As a result, it becomes more difficult for the glucose in the blood stream of the patient to diffuse through the cell wall into the cell. The blood glucose level slowly rises as the resistance slowly progresses and the pancreas is unable to continuously increase the production of insulin to adjust and normalize the progressively higher level of blood glucose. In addition to higher glucose levels resulting from insulin resistance, patients with Syndrome X have a higher risk for various diseases, including cardiovascular disease, pulmonary vascular disease, blood circulation disorder, peripheral vascular disease, peripheral neuropathy, renal disease and several other diseases commonly associated with diabetes.

The disease is considered to become Type II diabetes when the patient's blood glucose can no longer be maintained at normal levels. Once the disease has reached this point, the patient must be administered (1) oral hypoglycemic agents to increase the endogenous insulin production, or (2) supplemental insulin if or when endogenous insulin production is insufficient to normalize the blood glucose level.

As mentioned above, the theory set forth in this application, without limiting the present invention, is that relaxin has as much or more impact on diabetes as insulin. Current scholarship teaches that Type II diabetes is related solely to a deficit of insulin, but if that were true, then replacing the insulin in the patient would stabilize or reverse the progression of the disease. Unfortunately, that is not the case. Administering insulin to insulin dependent patients may blunt the progression of the disease, but the disease is not stabilized and continues to progress. Thus, while most physicians focus on a diabetic's lack of insulin, it is the complications that result from the progression of diabetes that incur the most deaths or serious physical problems for patients.

It is theorized herein that a relaxin deficit causes Type II diabetes. That is, the progressive deficit of the relaxin hormone causes a slow increase in insulin resistance in the patient and thereby causes the patient to slowly develop many of the symptoms of pre-diabetic syndrome or Syndrome X and ultimately causes the patient to develop Type II diabetes. The exact etiology of the relaxin deficit is unclear, but is possibly related to multiple risk factors. One of the risk factors appears to be obesity, which somehow affects either the production or utilization of relaxin, resulting in a gradual deficit of relaxin. Other factors include lack of exercise and poor diet.

It is further theorized that as the relaxin deficit progresses, other conditions also develop. The conditions may include such complications as cardiovascular disease, peripheral vascular disease, hypertension, renal disease, and lipid metabolism, and others. Relaxin has a role in all of the above disease processes. Relaxin deficit therefore initiates and intensifies these diseases. Treatment with relaxin may stabilize or reverse the progression of any of these diseases.

For example, as the relaxin deficit progresses, isolated cholesterol and or blood pressure may be elevated. There may be isolated perfusion problems within the cardiac vasculature resulting in cardiovascular disease, or circulatory problems in the lower extremities resulting in peripheral vascular disease. The severity of these diseases and the associated symptoms are related to the speed with which the relaxin deficit develops and the duration of the deficit.

The two hormone theory with respect to Type I diabetes is a different story, but the concept is the same. Unlike Type II, which originates from a resistance to insulin, Type I diabetes is initiated by an insulin deficit state that is created by the body's immune system attacking the body's own insulin production cells within the pancreas. In this situation, insulin is an antigen. After several years of suffering from this insulin deficit and externally supplementing the insulin levels in the body, the patient inevitably develops a state of relaxin deficit and the disease eventually has all the symptoms and indications of Type II diabetes. It is possible that Type I diabetes and the resulting state of insulin deficit burden the relaxin use within the body to the point that a relaxin deficit is produced. It is also possible that the immune response directed to the insulin as an antigen in the Type 1 diabetic may also attack the insulin-like relaxin molecule as well. Relaxin is an effective treatment of Type I diabetes because of the similarity of the molecular structures of relaxin and insulin. Thus, the two hormone deficit theory explains the origins of both Type I and Type II diabetes.

Despite the relevance of relaxin to these conditions, blood glucose level and the exogenous supplementation of insulin remain the staples of diagnostic parameters for both physicians and layman alike. And despite the focus on these parameters, Applicant submits as explained above that it would be equally useful, if not more useful, to additionally measure the level of relaxin in a patient to gauge the overall progression of diabetes within that individual.

In comparison to the relaxin deficit, it is theorized herein that the deficit of insulin is secondary to the diabetes disease process and is not the cause. At best, the insulin deficit serves to normalize the blood glucose level and plays a minimal role in the genesis of diabetes.

While not limited by theory, given the theory posed herein that diabetes is a two-hormone disease, a single molecule such as insulaxin that possesses both the hormonal activities of relaxin and insulin should be a highly effective treatment for diabetes, eliminating, arresting, or delaying the progression of the disease.

### Treatment of Alzheimer's Disease with Relaxin a Combination of Insulin and Relaxin, or Insulaxin

Relaxin, according to another embodiment, can be used to treat Alzheimer's disease. That is, administration of relaxin can reduce or halt the effects of Alzheimer's disease or even reverse the pathogenesis of the disease. In a further embodiment, relaxin can be administered to prevent the development of Alzheimer's in people predisposed to the disease.

Without being limited by theory, it is believed that relaxin is an effective treatment of Alzheimer's because relaxin can cross the blood brain barrier and reduce the insulin resistance of brain cells.

Recent studies have identified a link between diabetes and Alzheimer's. It has long been known that diabetes patients have an increased risk of being stricken with Alzheimer's later in life, though the mechanism could not be identified. However, some have suggested, based on the recently-identified link, that Alzheimer's may be triggered when brain cells cannot properly process sugar, just as Type 2 Diabetes is triggered when insulin loses its ability to properly process sugar. Further, scientists recently discovered that insulin and its related proteins are produced in the brain and that there is a link between Alzheimer's and lower-than-normal levels of the brain insulin and its proteins. More specifically, the studies indicated that lower-than-normal levels of brain insulin are associated with the onset of Alzheimer's. Researchers also determined that a drop in brain insulin production or brain insulin levels contributed to the degeneration of brain cells. Given the role of insulin in this phenomenon, some researchers have designated the condition “Type 3 Diabetes.”

In accordance with one embodiment, relaxin administered to a patient crosses the blood brain barrier and reduces the resistance of brain cells to insulin. As described above, studies have shown that relaxin decreases cell resistance to insulin. In one embodiment, the relaxin reduces the insulin resistance by binding with receptor sites in the brain and thereby reduces the brain cell resistance to the insulin. Thus, relaxin can be administered to a person predisposed to Alzheimer's to maintain the low insulin resistance of the patient's brain cells, thereby preventing the onset of Alzheimer's. Alternatively, relaxin can be administered to a patient suffering from Alzheimer's to reduce the insulin resistance of the patient's brain cells, thereby reducing, halting, or reversing the progress of Alzheimer's.

According to another embodiment of the present invention, both relaxin and insulin can be administered simultaneously to treat Alzheimer's patients, including Alzheimer's patients who exhibit abnormal blood glucose levels. That is, both polypeptides can be administered together in the same dosage. The combination of both polypeptides can have the same effect or, according to one aspect of the invention, a more powerful effect, with respect to treatment of Alzheimer's patients than either polypeptide alone.

In a further embodiment, Alzheimer's patients, including Alzheimer's patients with abnormal blood glucose levels, can be treated with insulaxin, as described above. In accordance with one aspect of the invention, treating Alzheimer's patients, including those with elevated blood glucose levels, with insulaxin can have the same effect or, according to one aspect of the invention, a more powerful beneficial effect than relaxin alone.

### Anti-Aging Treatment with Relaxin a Combination of Insulin and Relaxin, or Insulaxin

In a further embodiment, relaxin can be administered to non-diabetic individuals as a supplement to delay the aging process, improve health and longevity, maintain proper organ function, or maintain a youthful appearance.

The administration of relaxin is an effective anti-aging treatment because relaxin mimics the physiological effects of caloric restriction, which is the only previously known scientifically proven anti-aging treatment. Caloric restriction causes (1) decreased insulin levels, (2) decreased circulating free radical levels, (3) increased secretion of DHEA, and (4) decreased body temperature. Animal studies have shown that caloric restriction can extend an animal's life span by 40%. Without being limited by theory, the results of some studies have shown that insulin levels, along with growth hormone, may be factors in the life-extending effects of calorie restriction. Other studies have found a link between caloric restriction and the activation of an information regulator—Sir2.

Similarly, relaxin influences the insulin resistance of cells throughout the body, as explained above. The decrease in insulin resistance causes an associated reduction in circulating insulin levels. Further, relaxin also slows the oxidative process in the body, thereby decreasing circulating free radical levels. In addition, relaxin causes in an increase in the secretion of DHEA in the body by influencing the regulation of many of the body's glandular functions. Relaxin also helps regulate body temperature. In fact, most patients to which relaxin is administered exhibit a decrease in body temperature.

Thus, one method of the present invention relates to administration of relaxin to a patient, thereby producing the same or similar effects to caloric restriction, which in turn can have anti-aging benefits for a patient. Alternatively, relaxin and insulaxin can be administered to a patient. In a further alternative, insulaxin can be administered. In yet another alternative, any polypeptide related to relaxin can be administered as an anti-aging treatment.

### Hormone Replacement Therapy and Treatment of Menopause and Related Conditions

Relaxin, according to one embodiment, may be used in hormone replacement therapy (“HRT”) to treat many of the conditions relating to menopause in women. In one aspect of the invention, relaxin alone can be administered. Alternatively, relaxin can be administered in combination with either estrogen or progesterone or both.

In a further embodiment, relaxin may be administered to a patient alone or in combination with either estrogen or progesterone or both to delay the onset of conditions relating to menopause, such as cardiovascular disease, adult onset diabetes, hypertension, high cholesterol, osteoarthritis, bone weakness or brittleness or osteoporosis, central nervous system (“CNS”)-related conditions, including sleep loss, irritability, memory loss, depression, etc., and many other disease processes that many women begin to experience or begin to develop during the menopausal period.

As discussed above, “relaxin” encompasses the relaxin hormone, related polypeptides, relaxin-like factors, analogs, and all other molecules described above. Further, administration of relaxin or relaxin in combination with either estrogen or progesterone or both can occur by any administration method and in any form of administration as explained above.

Without being limited by theory, relaxin may be beneficial in HRT because it is the third hormone in the menstrual cycle—along with estrogen and progesterone, and thus it is logical that it would be an effective component of HRT. Unfortunately, physicians have attributed every single menopause symptom to an estrogen deficit or occasionally to a progesterone deficit. Since the majority of physicians do not recognize that three hormones play a role in a woman's menstrual cycle, most do not realize that relaxin may play a role in many of these menopause-related problems, including memory recall, irritability, vasomotor changes and various other known problems that are commonly associated with menopause. Thus, it is theorized that menopausal difficulties may be related to a relaxin deficit or a combined deficit of the three major hormones. As a result, administering relaxin becomes as important as the other two hormones in a post-menopausal woman who complains of problems related to menopause.

In addition, without being limited by theory, human relaxin may be beneficial in HRT because it is bio-identical to the hormones produced by women. HRT has been rather controversial lately because of the findings that estrogen can promote multiple medical problems in women such as breast cancer, stroke, cardiovascular disease, and possibly uterine cancer. These findings have caused unease among many women regarding the safety of HRT. Without being limited by theory, it has been theorized that the medical problems resulting from HRT may be caused by the fact that two common HRT treatments—Premarin, which is conjugated estrogen, and Provera, which is a synthetic form of progestin—are not bio-identical to the hormones that are produced in the female body. That is, the synthetic and conjugated hormones are not identical to the hormones produced in the female body. Administration of such synthetic molecules and their extremely active metabolites produces activities in the patient above and beyond the normal activities of the bio-identical hormones. These additional activities may have adverse effects in patients on such HRT. In contrast, human relaxin is bio-identical to the hormones produced in the female body. Because it does not have any active metabolites, human relaxin may reduce the number of side effects experienced as a result of HRT.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

Although the present invention has been described with reference to preferred embodiments, persons skilled in the art will recognize that changes may be made in form and detail without departing from the spirit and scope of the invention.

What is claimed is:

1. A method for treating diabetes-related conditions in a subject, comprising:
administering relaxin to the subject, whereby the subject's supplemental insulin requirements are reduced.

2. The device of claim 1 wherein the method further comprises administering insulin.

3. The device of claim 2 wherein the administering insulin comprises administering a form of insulin chosen from the group consisting of human insulin, porcine insulin, ovine insulin, and bovine insulin.

4. The device of claim 1 wherein the administering relaxin comprises administering a hybrid molecule of relaxin and insulin.

5. The device of claim 1 wherein the subject has a condition chosen from the group consisting of pre-diabetic syndrome, Type II diabetes, Type I diabetes, and Alzheimer's disease.

6. The method of claim 1 wherein the administering relaxin to the subject results in decreased insulin resistance in the subject.

7. The method of claim 1 wherein the administering relaxin to the subject results in a delay in the onset of insulin resistance in the subject.

8. The method of claim 1 wherein the administering relaxin to the subject results in decreased levels of glucose in the subject's blood.

9. The method of claim 1 wherein the administering relaxin to the subject results in decreased levels of Hb1Ac in the subject's blood.

10. The method of claim 1 wherein the administering relaxin to the subject results in improvements in the subject's condition relating to diabetes.

11. The method of claim 1 wherein the subject has pre-diabetic syndrome and the administering relaxin to the subject results in a delay in an onset of clinical diabetes.

12. The method of claim 1 wherein the subject has clinical Type II diabetes and the administering relaxin to the subject results in a delay in an onset of a more advanced state of the clinical Type II diabetes.

13. The method of claim 1 wherein the subject has clinical Type I diabetes and the administering relaxin to the subject results in a delay in an onset of a more advanced state of the clinical Type I diabetes.

14. The method of claim 1 wherein the subject has clinical Type I diabetes and the administering relaxin to the subject results in suppression of an immuno-response to insulin.

15. The method of claim 1 wherein administering relaxin to the subject results in a decrease in the incidence of disease related to diabetes.

16. The method of claim 15 wherein the disease related to diabetes is a disease chosen from the group consisting of cardiovascular disease, heart disease, renal disease, circulatory disease, peripheral vascular disease, retinopathy, fibrosis pulmonary hypertension, elevated blood pressure, bowel problems, rapid aging, organ deterioration, bone weakness, bone brittleness, osteoporosis, and degenerative joint disease.

17. The method of claim 16 wherein the elevated blood pressure is related to hypertension.

18. The method of claim 16 wherein the bowel problems are chosen from the group consisting of atony and dysautonomia.

19. The method of claim 1 wherein the administering relaxin to the subject results in decreased insulin resistance in brain cells of the subject.

20. The method of claim 1 wherein the administering relaxin to the subject results in improvements in the subject's condition relating to Alzheimer's disease.

21. The method of claim 1 wherein the subject has Alzheimer's disease and the administering relaxin to the subject results in a delay in an onset of Alzheimer's disease.

22. The method of claim 1 wherein the subject is predisposed to Alzheimer's disease and the administering relaxin to the subject results in prevention of Alzheimer's disease.

23. A method for treating menopause-related conditions in a subject, comprising:
administering relaxin to the subject, whereby the subject's menopause-related conditions is improved.

24. The method of claim 23 wherein the administering relaxin is a part of a hormone replacement therapy.

25. The method of claim 23 wherein the method further comprises administering estrogen.

26. The method of claim 23 wherein the method further comprises administering progesterone.

27. The method of claim 23 wherein the method further comprises administering estrogen and progesterone.

28. The method of claim 23 wherein the administering relaxin to the subject results in a delay in the onset of conditions related to menopause.

29. The method of claim 28 wherein the conditions related to menopause are selected from the group consisting of cardiovascular disease, adult onset diabetes, hypertension, high cholesterol, bone weakness, bone brittleness, osteoporosis, degenerative joint disease, and CNS-related conditions.

30. The method of claim 29 wherein the CNS-related conditions are selected from the group consisting of sleep loss, memory loss, and depression.

31. The method of claim 23 wherein the administering relaxin to the subject results in an improvement in conditions of the subject related to menopause.

32. The method of claim 31 wherein the conditions related to menopause are selected from the group consisting of cardiovascular disease, adult onset diabetes, hypertension, high cholesterol, bone weakness, bone brittleness, osteoporosis, degenerative joint disease, and CNS-related conditions.

33. The method of claim 31 wherein the CNS-related conditions are selected from the group consisting of sleep loss, memory loss, and depression.

34. A method of anti-aging treatment, comprising:
administering relaxin to a subject, whereby the subject's insulin resistance is decreased.

35. A method for treating diabetes-related conditions in a subject, comprising suggesting administration of relaxin to the subject.

36. The method of claim 35, further comprising administering relaxin to the subject, whereby the subject's supplemental insulin requirements are reduced.

37. The method of claim 35, further comprising suggesting administration of insulin.

38. The method of claim 37, further comprising administering insulin to the subject.

39. A kit for treating a condition in a subject, the kit comprising:
(a) a dosage of relaxin; and
(b) instructions for administration of the relaxin.
